(19) United States
(12) Patent Application Publication
(10) Pub. No.: US 2026/0272595 A1
(43) Pub. Date: Sep. 17, 2026

(54) PRESSURE MONITORING SYSTEM FOR TOURNIQUET AND METHOD OF USE THEREOF

(71) Applicant: MY01 IP Holdings Inc., Montréal (CA)

(72) Inventors: Olivier Bataille, Beloeil (CA); Anya Jesson, Montréal (CA); Christopher Hugh Jolly, Lexington, CA (US); Eric Schaal, Montréal (CA); René Gagner, Montréal (CA)

(21) Appl. No.: 19/403,166

(22) Filed: Nov. 27, 2025

Related U.S. Application Data
(60) Provisional application No. 63/772,409, filed on Mar. 14, 2025.

(51) Int. Cl. A61B 90/00; A61B 17/132

(52) U.S. Cl. CPC A61B 90/06; A61B 17/1322; A61B 2090/065

(57) ABSTRACT

A pressure monitoring system for recording pressure applied to a limb by a tourniquet; it has a pressure sensor for measuring pressure applied by tourniquet to the limb; a power source; a processor; an input/output interface; memory including program code that, when executed by the processor, causes the processor to: receive pressure readings from the pressure sensor; upon receiving a trigger event, generate time readings indicative of a passing of time following the trigger event; and cause a transmission of the received pressure readings and the time readings for one or more of: storage of the received pressure readings and the time readings; and display, on a display, information on pressure applied to the limb by the tourniquet over time related to historical pressure-time data corresponding to a relation between the time readings and the pressure readings over a period of time; a method of use thereof.

The present application claims priority from U.S. provisional patent application No. 63/772,409 filed on Mar. 14, 2025, incorporated herein by reference.

### TECHNICAL FIELD

The present disclosure relates to tourniquets, and more particularly to accessory systems to be used alongside tourniquets.

### BACKGROUND

A tourniquet is used to create ischemia within the limb, reducing the flow of blood to that limb. The tourniquet may be used following an emergency (such as an injury), a surgery, may have post-operative use, etc.

In some scenarios, such as when treating soldiers on a battlefield, or when treating athletes during a sports competition, first responders may be present on the scene to stabilize the patient, in view of transferring the patient to a healthcare provider who can perform more extensive treatment on the patient (e.g. perform surgery).

However, when the healthcare provider receives the patient, information on the state of the patient, including on the application of the tourniquet to the patient by the first provider, may be lacking to properly assessing the nature of the medical procedure to adopt for the patient (e.g. performing an operation to salvage the limb, taking the decision to amputate the limb. etc.), as the tourniquet was applied by the first responder and not the healthcare provider. The proper application of the tourniquet by the first responder can influence the decision of the medical provider vis-à-vis the patient. The healthcare provider may struggle in selecting the appropriate course of action for the patient without benefitting from information on how the tourniquet had been applied to the limb of the patient.

### SUMMARY

Applying circumferential compression or occlusion pressure to the limb by the tourniquet that is manipulated by a first responder that is of a given target pressure value may be important for preserving the limb, where application of a wrong pressure may damage the limb, and in some situations, along with other factors, may result in the healthcare provider having no other option but to amputate the limb. Therefore, data on the pressure applied by the tourniquet to the limb, as handled by the first responder, becomes important in deciding upon the course of action to be taken by the healthcare provider regarding treatment of the limb.

The present disclosure relates to a pressure monitoring system to be used with a tourniquet, for measuring pressure applied by the tourniquet to a limb of a patient, the tourniquet handled by the first responder, and for communicating information on the pressure that has been applied by the tourniquet to a healthcare provider who is responsible for deciding upon the appropriate medical course of action to take with respect to the limb of the patient, that course of action influenced by a determination that the tourniquet was properly applied by the first responder onto the limb.

The pressure monitoring system includes a pressure sensor for insertion between the tourniquet and the limb (e.g. may have a coin or disc shape), squeezed between the tourniquet and the limb, for measuring the pressure applied by the tourniquet to the limb.

The pressure monitoring system includes a controller or computer module for receiving the pressure readings from the pressure sensor, storing the pressure readings either locally (on local memory) or remotely (at a remote server) with time readings (providing information regarding when the pressure reading was taken with respect to other pressure readings). This stored information may then be communicated to the healthcare provider when the patient, with the tourniquet, is transferred to the healthcare provider, as historical time-pressure data. For instance, in some implementations, a trauma team at a healthcare establishment who is entrusted with a patient having received a tourniquet benefits from the data shared from the monitoring system on the patient, this data providing the trauma team with patient data that can assist the team in deciding upon a course of action to treat the patient.

A broad aspect is a pressure monitoring system for recording pressure applied to a limb by a tourniquet. The system includes a pressure sensor for insertion between the tourniquet and the limb, and for measuring pressure applied by tourniquet to the limb; a power source; a processor; an input/output interface; memory including program code that, when executed by the processor, causes the processor to: receive pressure readings from the pressure sensor; upon receiving a trigger event, generate time readings indicative of a passing of time following the trigger event; and cause a transmission, via the input/output interface, of the received pressure readings and the time readings for one or more of: storage of the received pressure readings and the time readings; and display, on a display, information on pressure applied to the limb by the tourniquet over time related to historical pressure-time data corresponding to a relation between the time readings and the pressure readings over a period of time.

In some embodiments, the memory further may include program code for causing the processor to store the received pressure readings in relation to the generated time readings in the memory as the historical pressure-time data; and upon an occurrence of a trigger event, cause a transmission, via the input/output interface, of the historical pressure-time data for display on the display of information on pressure applied to the limb by the tourniquet over time related to historical pressure-time data, wherein the causing of a transmission occurs upon an occurrence of a trigger event.

In some embodiments, the system may include the display.

In some embodiments, the memory may further include program code for causing the processor to display on the display real time pressure information corresponding to the received pressure readings and a time value corresponding to the time readings.

In some embodiments, the system may include a user input interface, wherein the trigger event may be receiving input received at the user input interface.

In some embodiments, the information on pressure applied to the limb by the tourniquet over time related to historical pressure-time data may be displayed on the display.

In some embodiments, the user input interface may be a button.

In some embodiments, the display may be a display of an external computing device, and wherein the trigger event may be a reception of a data request received from the external computing device.

In some embodiments, the input/output interface may be configured to establish a wireless connection with the external computing device.

In some embodiments, the input/output interface may be configured to establish a wired connection with the external computing device.

In some embodiments, the wired connection may be a USB connection.

In some embodiments, the display may be a display of an external computing device, and wherein the trigger event may be a detection of the external computing device being in range of the system.

In some embodiments, the transmission, via the input/output interface, of the received pressure readings and the time readings may be caused to be transmitted to an external server, and wherein the storage of the received pressure readings and the time readings may be performed in the external server for querying by a healthcare provider.

In some embodiments, the pressure sensor may be a force-sensing resistor (FSR).

In some embodiments, the pressure sensor may be a force-sensing capacitor.

In some embodiments, the trigger event may correspond to a beginning of an application of pressure to the limb using the tourniquet.

In some embodiments, the system may include a timer user input interface, wherein the trigger event corresponding to a reception of user input may be provided via the timer user input interface.

In some embodiments, the timer user input interface may include a button.

In some embodiments, the system may include a sheath for receiving and protecting from fluids the pressor sensor, the power source, the input/output interface, the processor and the memory.

In some embodiments, the program code may further cause the processor to cause a generation of a signal when a target pressure applied by the tourniquet on the limb is reached, determined from the pressure readings received from the pressure sensor.

In some embodiments, the signal may include a visual indication appearing on a display.

In some embodiments, the signal may be a sound, where the system may include a speaker for generating the sound.

In some embodiments, the system may include the tourniquet.

In some embodiments, the input/output interface, the processor and the memory may be contained within a housing that is attached to the tourniquet.

Another broad aspect is a method of sharing information on a state of a limb that has undergone ischemia resulting from the application of a tourniquet to the limb. The method includes upon occurrence of a trigger event, receiving historical pressure-time data generated by a pressure sensor adapted to generate pressure readings of pressure applied from the tourniquet to the limb, the historical pressure-time data indicative of pressure applied over a period of time by the tourniquet, wherein the historical pressure-time data provides information on the state of the limb based on a sufficiency of pressure applied to the limb by the tourniquet.

In some embodiments, the trigger event may be a reception or transmission of user input for causing transmission of the received historical pressure-time data.

In some embodiments, the information on the state of the limb may be displayed as a graph corresponding to the historical pressure-time data.

In some embodiments, the information on the state of the limb may be displayed as an average pressure value over the period of time.

In some embodiments, the historical pressure-time data may be received over a short-range wireless connection from a computer module receiving the pressure readings from the pressure sensor, the computer module joined to the tourniquet.

In some embodiments, the historical pressure-time data may be received over a wired connection from a computer module receiving the pressure readings from the pressure sensor, the computer module joined to the tourniquet.

Another broad aspect is a method of sharing information on a state of a limb that has undergone ischemia resulting from the application of a tourniquet to the limb. The method includes, upon occurrence of a trigger event, causing a display of information indicative of the state of the limb based on a sufficiency of pressure applied to the limb by the tourniquet over a period of time, the information derived from historical pressure-time data generated by a pressure sensor adapted to generate pressure readings of pressure applied from the tourniquet to the limb, to, the historical pressure-time data indicative of pressure applied over the period of time by the tourniquet.

In some embodiments, the information may be displayed on a display connected to the tourniquet.

In some embodiments, the method may include transmitting the historical pressure-time data to an external computer following the trigger event, and wherein the information is displayed on a display of the external computer.

In some embodiments, the trigger input may be a reception of user input to cause the display of information.

In some embodiments, the information may be displayed as a graph of pressure as a function of time.

In some embodiments, the method may include fastening the pressure sensor to the tourniquet using a clip.

In some embodiments, the clip may include teeth for digging into the tourniquet.

Another broad aspect is a pressure monitoring system for recording pressure applied to a limb by a tourniquet. The system includes a pressure sensor for insertion between the tourniquet and the limb, and for measuring pressure applied by tourniquet to the limb; a power source; a processor; an input/output interface; memory including program code that, when executed by the processor, causes the processor to: receive pressure readings from the pressure sensor; upon receiving a trigger event, generate time readings indicative of a passing of time following the trigger event; and cause a transmission, via the input/output interface, of the received pressure readings and the time readings for one or more of: storage of the received pressure readings and the time readings; and display, on a display, information on pressure applied to the limb by the tourniquet over time related to historical pressure-time data corresponding to a relation between the time readings and the pressure readings over a period of time; and a housing for containing at least the processor and the memory.

In some embodiments, the display may be a liquid-crystal display.

In some embodiments, the user input interface may include two buttons, wherein a first button of the two buttons may be located on a first side of the housing and a second of the two buttons may be located on a second side of the housing opposite the first side of the housing, and wherein the trigger event may be user input received at the first button and the second button.

In some embodiments, the trigger event may correspond to a beginning of an application of pressure to the limb using the tourniquet.

In some embodiments, the system may include a timer user input interface, wherein the trigger event corresponding to a reception of user input may be provided via the timer user input interface.

In some embodiments, the system may include a fastener that is a clip located on the housing for receiving the tourniquet.

In some embodiments, the clip may include one or more teeth projecting from a surface of the clip that is facing the housing and that is adapted to contact the tourniquet once the tourniquet is inserted in a space defined between the clip and the housing.

In some embodiments, the one or more teeth may include rows of teeth.

### BRIEF DESCRIPTION OF THE DRAWINGS

The invention will be better understood by way of the following detailed description of embodiments of the invention with reference to the appended drawings, in which:

FIG. 1A is a photograph of an exemplary monitoring system for recording pressure applied to a limb by a tourniquet;

FIG. 1B is a photograph of the exemplary pressure monitoring system of FIG. 1A joined to an exemplary tourniquet;

FIG. 1C is a photograph of the exemplary pressure monitoring system and tourniquet of FIG. 1B;

FIG. 1D is a photograph of the exemplary pressure monitoring system of FIG. 1A and of an exemplary tourniquet, the pressure monitoring system connected to an external computing device through a wired connection;

FIG. 2A is a block diagram of an exemplary monitoring system;

FIG. 2B is a block diagram of exemplary network architecture for monitoring the pressure applied by a tourniquet to a limb of a patient;

FIG. 3 is a flowchart diagram of an exemplary method for monitoring pressure applied to a patient via a tourniquet and sharing information on the applied pressure with a healthcare provider;

FIG. 4A is a drawing of a front view of an exemplary monitoring system for recording pressure applied by a tourniquet;

FIG. 4B is a drawing of a back view of the exemplary system of FIG. 4A;

FIG. 4C is a drawing of a right view of the exemplary system of FIG. 4A; and

FIG. 4D is a drawing of a left view of the exemplary system of FIG. 4A.

### DETAILED DESCRIPTION

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

From the foregoing it will be appreciated that, although specific embodiments have been described herein for purposes of illustration, various modifications may be made without deviating from the spirit and scope of the teachings. Accordingly, the claims are not limited by the disclosed embodiments.

### Definitions

In the present disclosure, by “patient” or “subject”, used interchangeably herein, it is meant a mammal, such as a human. The term “subject” or “patient” should not bring on any limitations as to the sex or age.

The term “treat”, “treating” or “treatment”, as used herein, refers to alleviating or improving the outcome of the subject with regard to a given disease or disorder, which may be quantifiable by improving at least one physical parameter, ailment or biomarker of the subject with the disease or disorder.

The term “reading” or “readings”, as used herein, refers to a representation of data collected from a device which may be a numerical value, a graph, a trendline, etc.

### Exemplary Monitoring System for Recording Pressure Applied to a Limb by a Tourniquet

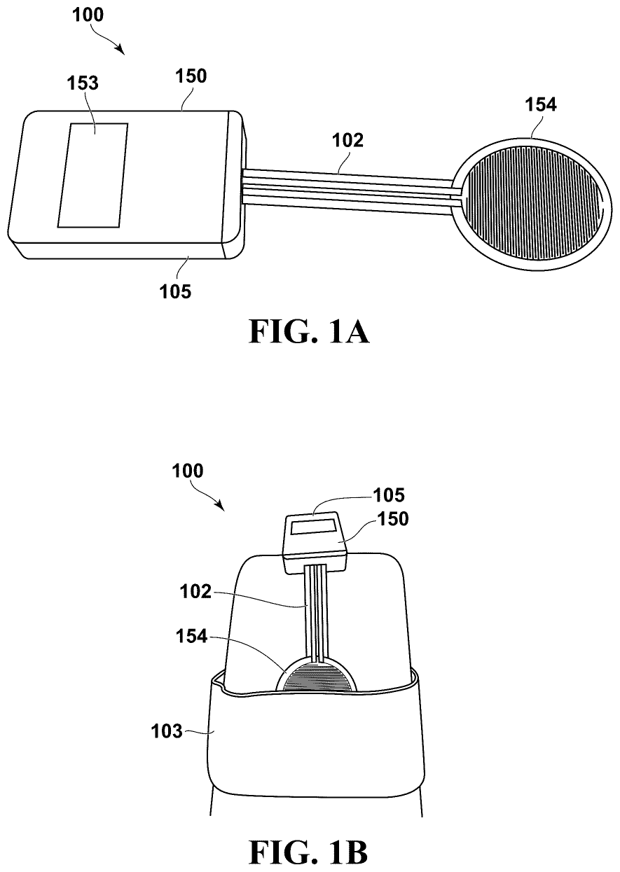

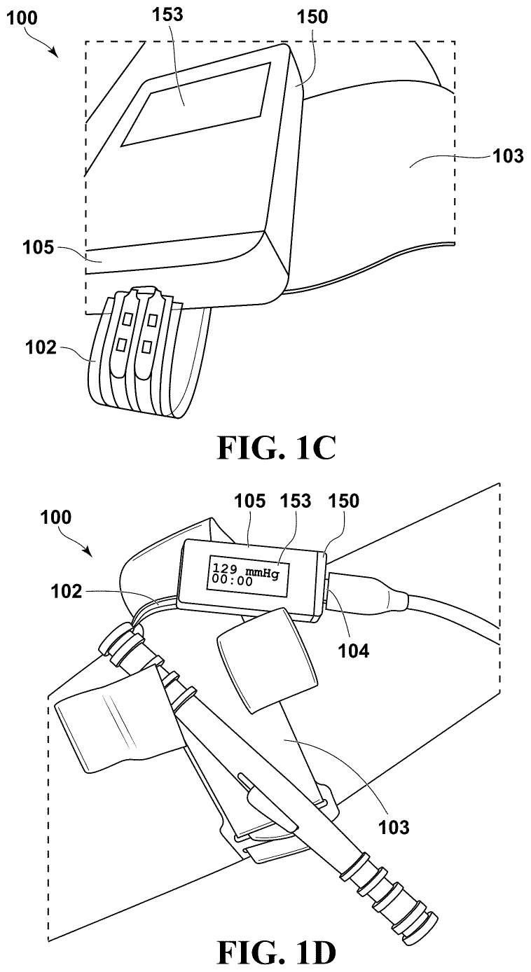

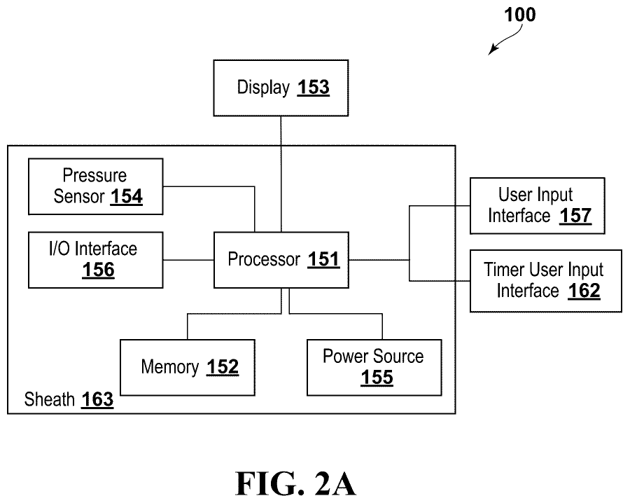

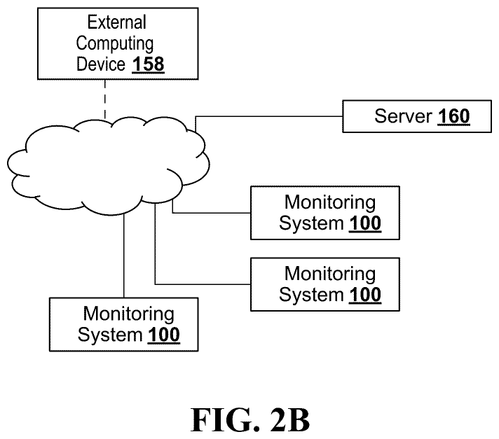

Reference is made to FIGS. 1A and 2A, illustrating an exemplary monitoring system 100 for recording pressure applied to a limb of a patient by a tourniquet 103.

The monitoring system 100 includes a computing device 150 and a pressure sensor 154. The computing device 150 is enclosed within a housing 105. The computing device 150 includes a processor 151 and memory 152. The computing device 150 may include a microprocessor.

An exemplary monitoring system 100 is illustrated being deployed with a tourniquet 103 in FIG. 1B. FIG. 1C illustrates the monitoring system 100 attached to the tourniquet 103 applied to a model of a limb of a patient.

The monitoring system 100 includes an input/output interface 156. The monitoring system 100 includes a power source 155.

The monitoring system 100 may include a user input interface 157.

The monitoring system 100 may include a timer user input interface 162.

The monitoring system 100 may include the tourniquet 103.

The computing device 150 may include a display 153 for, e.g., displaying real time pressure readings received from the pressure sensor 154, displaying time readings (e.g. generate by a timer), displaying information on pressure applied to the limb by the tourniquet 103 over time, displaying time values corresponding to time readings related to pressure applied to the limb by the tourniquet 103, etc.

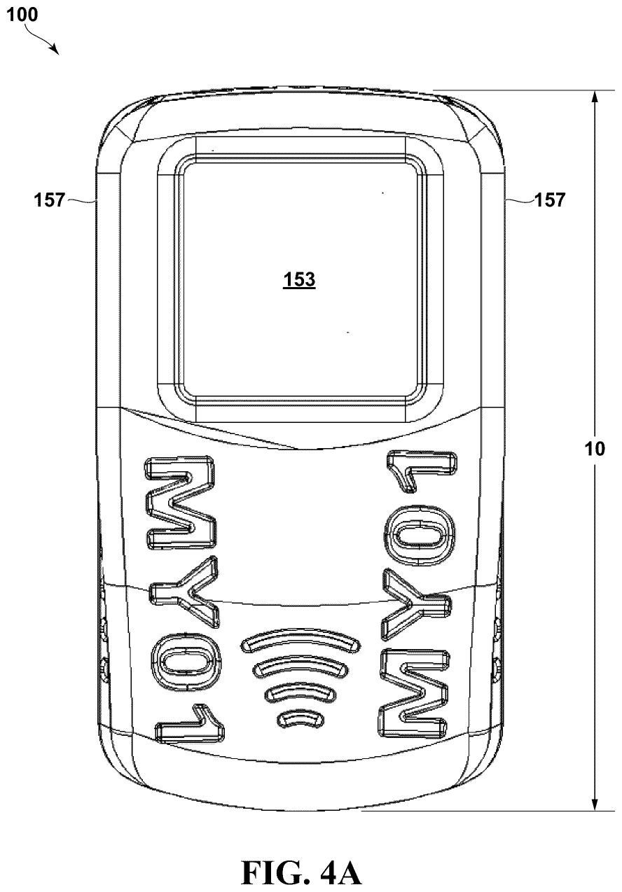

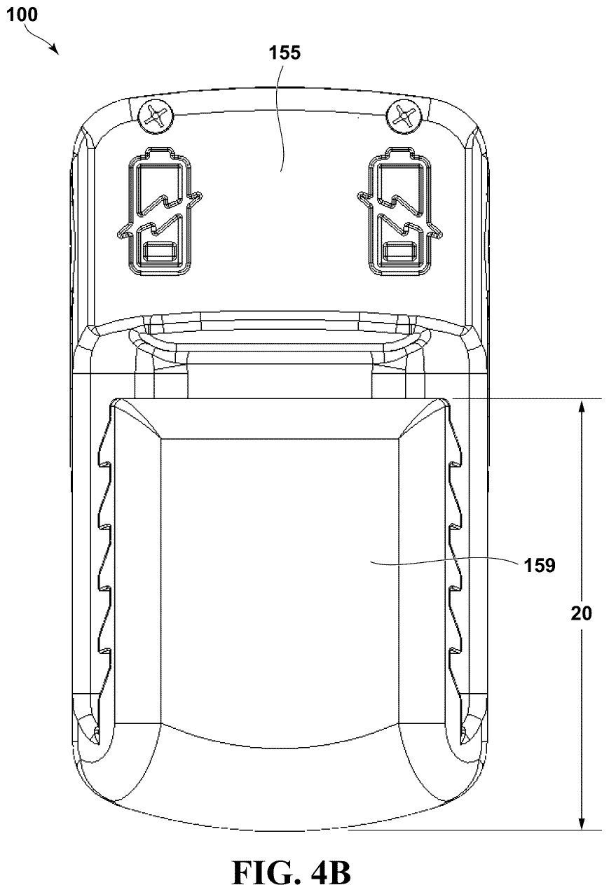

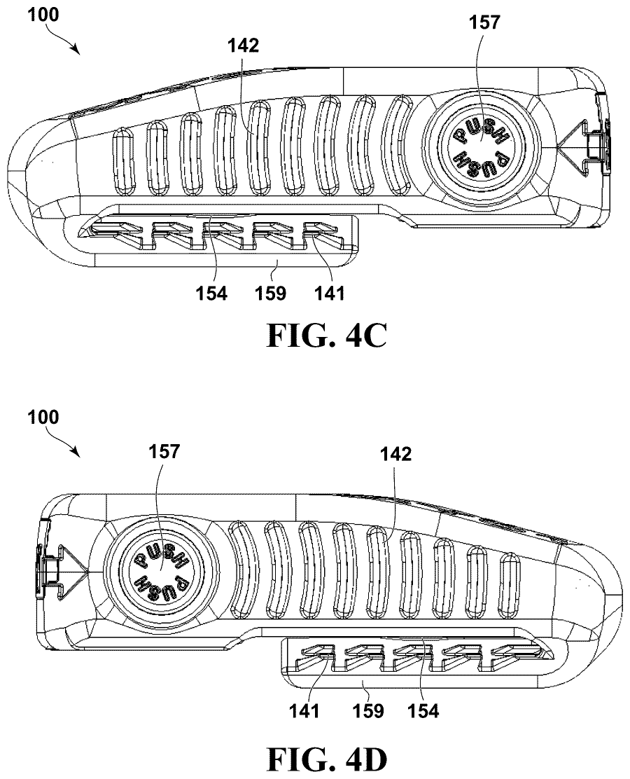

As shown in FIGS. 4A-4D, the housing 105 may include, on one or more sides of the housing 105, ribs or undulations 142 to improve a grip of a user of the computing device 150.

In some implementations, as illustrated in FIGS. 4A-4D, the computing device 150 may include a fastener 159 (e.g. a clip), joined to or integral with the housing 105, for enabling the housing 105 to secure to the tourniquet 103. The tourniquet 103 may fit between an underside of the computing device 150 and a surface of the fastener 159 facing the underside of the computing device 150. The pressure sensor 154 may be connected or integrated into the underside of the computing device 150. When the tourniquet 103 is secured to the computing device 150 using the fastener 159, the pressure sensor 154, connected to the underside of the computing device 154, receives a force applied by the tourniquet 103 onto the pressure sensor 154 as the tourniquet 103 is tightened through use of the tourniquet 103 when secured to a limb of a subject. The connecting of the pressure sensor 154 to the computing device 150 (e.g. underside of the computing device 150) and the use of the fastener 159 to fasten the computing device 150 to the tourniquet 103 enables the positioning of the pressure sensor 154 with respect to the tourniquet 103, such that a force applied by the tourniquet 103 is accurately read by the pressure sensor 154.

The fastener 159 may include one or more teeth 141 (or one or more rows of teeth 141) located on a surface of the fastener 159 facing an underside of the computing device 150 that are adapted to connect and dig into the tourniquet 103, further securing the computing device 150 to the tourniquet 103.

When the computing device 150 is joined to the tourniquet 103, a length 10 of the computing device 150 may be orthogonal with a length of the tourniquet 103. A length 20 of the fastener 159 may be parallel with the length 10 of the computing device 150.

The pressure sensor 154 is configured to measure pressure applied to a limb of a patient by a tourniquet. The pressure sensor 154 may be inserted between the limb and the tourniquet 103. The pressure may have a flat, coin or disk shape. The pressure sensor 154 may be a force sensing resistor (FSR) where force measurements obtained by the FSR may be converted into pressure readings by using an area value. The resistance of the FSR may change according to the force applied by the tourniquet to the limb, which can be measured with a voltage divider circuit. The pressure sensor 154 may also be a force sensing capacitor where the capacitance changes in response to an applied force. The force measurements obtained by the force sensing capacitor may be converted into pressure readings by using an area value. Force sensing capacitor may have a similar form factor as an FSR. It should also be understood that a pressure sensor 154 may also be any sensor capable of measuring a force or pressure applied to a limb by a tourniquet without departing from the present teachings. The pressure sensor 154 may be joined to a surface of the underside of the computing device 150.

The power source 155 may be or include secondary accumulator devices, such as rechargeable batteries (Nickel-Cadmium (NiCd), Lithium-Ion Batteries (Li-ion), etc.), batteries, supercapacitors, etc. Secondary accumulator devices may be continuously replaced by other batteries and recharged repeatedly. The power source 155 may also be AC electrical mains where the computing device 150 may be connected to the power source 155 through a wired connection. The microprocessor, or the processor 151 and memory 152, may be referred to herein as a controller. The power source 155 may be adapted for wireless charging (e.g. the power source 155 may be coupled to a wireless antenna receiver for receiving wireless power from an external source).

The computing device 150 may be a dongle, a portable computer as shown in FIGS. 4A-4D, etc.

The processor 151 may be a general-purpose programmable processor.

The computer readable memory 152 stores program instructions and data used by the processor 151. The computer readable memory 152 may also store pressure readings, time readings, historical pressure-time data, etc. The memory 152 may be non-transitory. The computer readable memory 152, though shown as unitary for simplicity in the present example, may comprise multiple memory modules and/or caching. In particular, it may comprise several layers of memory such as a hard drive, external drive (e.g. SD card storage) or the like and a faster and smaller RAM module. The RAM module may store data and/or program code currently being, recently being or soon to be processed by the processor 151 as well as cache data and/or program code from a hard drive. A hard drive may store program code and be accessed to retrieve such code for execution by the processor 151 and may be accessed by the processor 151 to store and access data. The memory 152 may have a recycling architecture for storing, for instance, pressure readings, time readings, historical pressure-time data etc., where older data files are deleted when the memory 152 is full or near being full, or after the older data files have been stored in memory 152 for a certain time.

The I/O interface 156 is in communication with the processor 151. The I/O interface 156 may include a network interface and may be a wired (through cable 102 and/or 104) or wireless interface for establishing a connection with the external computing device 158 or a remote server 160, and optionally, transmit pressure readings, time readings, historical pressure-time data and/or other data to the external computing device 158 or remote server 160.

In some implementations, the I/O interface 156 may be adapted to transfer data to an external computing device 158 (e.g. a smartphone, a smartwatch, a tablet computer, etc.) that is in proximity with the I/O interface 156 (e.g. through near-field communication, a short-range wireless communication such as a Bluetooth™ connection, etc.) In some instances, a security protocol may be implemented prior to or during transfer of data wirelessly from the system 100 to the external computing device 158 (e.g. such as a handshake, encryption of data employing an asymmetric key, etc.)

The processor 151, the memory 152 and the I/O interface(s) 156 may be linked via bus connections.

The user input interface 157 is a device (e.g. a component) through which the user may provide input to the computing device 150. A user input interface 157 may be, or include, a button, a mouse, a keyboard, a joystick, a controller, a touchscreen (e.g. of display 153), a microphone (for capturing speech or sounds from the user), a motion detector, etc. The user input interface 157 may be integrated into the housing 105 of the computing device 150.

As illustrated in FIGS. 4A-4D, the user input interface 157 may include two buttons or sub-interfaces. Each of the two buttons may be positioned on a separate side of the computing device 150, where both buttons 157 are to be pressed simultaneously (e.g. by the thumb and a finger of the user) in order to cause the computing device 150 to perform a given action. The configuration of the computing device 150 to require the pressing of both buttons 157 may act as a failsafe, thereby preventing accidental pressing of a button of the user input interface 157 that would cause the initiation of an unwanted action by the computing device 150, which may occur, for instance, in intensive settings, such as when applying a tourniquet on a battlefield.

The program code stored in memory 152 of the computing device 150, when executed by the processor 151, may cause the processor 151 to carry out different actions depending on the nature of the user input received at the user input interface 157. For instance, receipt a long press (at the two buttons 157) may cause a performance of a given effect, while a sequence of short presses at the two buttons 157 may cause the performance of a different effect. In one embodiment, receipt of a long press of both buttons 157 may cause the processor 151 to initiate a mode where the processor 151 begins to store (and display on display 153) readings received from the pressure sensor 154 (e.g. a live mode). Receipt of sequential presses (e.g. three sequential presses) at the two buttons 157 may instead cause the processor 151 to initiate a switch of the computing device 150 into a dormant mode, where the pressure readings generated by the pressure sensor 154 are being recorded and stored in memory 152 of the computing device 150, but are not displayed on display 153 in order to conserve power of the power source 155. It will be understood that the actions performed by the computing device 150 in response to a given user input type received at the user input interface 157 may vary without departing from the present teachings.

In some instances, the user input interface 157 may include an additional interface for causing the system 100 to restart the timer and erase from memory prior data readings (e.g. pressure readings and/or temperature readings) (i.e. a reset of the system 100) when user input is received at this additional interface. Such user input at the additional interface may be received when the system 100 is to be used with a new patient, or at a different time for the existing patient (e.g. to establish a new condition or state for that patient). For instance, the additional interface may be a button recessed within a well located in the housing 105 of the computing device 150, adapted to receive a narrow object, such as a pin or a tip of a wire. The additional interface may be positioned on the computing device 150 in order to increase the difficulty of providing user input at the additional interface (e.g. to not accidentally cause a reset of the system 100). It will be understood that the nature and shape of the additional interface may vary without departing from the present teachings.

The timer user input interface 162 is a device through which the user may provide input to the computing device 150 to start or stop a timer for taking time readings. The time user input interface 162 may be, or include, a button, a mouse, a keyboard, a joystick, a controller, a touchscreen (e.g. of display 153), a microphone (for capturing speech or sounds from the user), a motion detector, etc. It will be understood that the user input interface 157 may operate as the timer user interface 162, or that the timer user input interface 162 may be a user input interface (e.g. one or more buttons) of the user input interface 157.

The display 153 is a screen for sharing information to the user (e.g. real time pressure readings received from the pressure sensor 154, time values corresponding to time readings generated by a clock or timer, information on pressure applied to the limb by the tourniquet over time, time values corresponding to time readings related to pressure applied to the limb by the tourniquet, an alert that a certain pressure has been applied to the limb, a warning that insufficient pressure has been applied to the limb, a warning that a certain period of time has passed since a trigger event, a graphical representation of historical pressure-time data, etc.) The display 153 may be a screen for a computer or dongle, a touchscreen (where the display 153 may also act as a user input interface 157 or timer user input interface 162), etc. In some embodiments, the display 153 may be attached to or integrated to the pressure sensor 154 and/or computing device 150, however the display 153 may also be an external device or part of an external computing device. When the display 153 is attached to the pressure sensor and/or computing device 150, the display 153 may be secured onto the skin of the patient or onto the tourniquet applied to the limb of the patient by an adhesive. The display 153 may be integrated into the housing 105.

In some instances, the display 153 may be a screen that does not emit light, or emits only a little light, e.g. in order to avoid alerting enemy military forces when applying the tourniquet and using the system 100 on a battlefield. Exemplary screen types may include, but are not limited to, liquid-crystal displays, a screen with a filter for filtering out certain frequencies of light, an electronic paper screen, etc.

In some embodiments an external computing device 158 may display information from a plurality of monitoring systems 100. An external computing device 158 displaying information from a plurality of monitoring systems 100 may be used to monitor the pressure applied to limbs of a single patient or multiple patients.

In some instances, the system 100 may include an alert emitter (not shown) to cause the generation of an alert (e.g. a speaker for generating a sound, an accelerometer for generating a vibration, the display 153 adapted to produce a message on the graphical user interface appearing on the display 153, etc.) The program code stored in memory 152 may cause the processor 151 to start a timer function when pressure is first applied by the tourniquet onto a limb of a patient (e.g. when readings generated by the pressure sensor 154 are greater than a defined threshold value, indicative that the tourniquet is being applied to a limb of a subject). When the timer reaches a given measured time (e.g. an hour), the processor 151 may cause the alert emitter to generate the alert, e.g., to the subject using the tourniquet, that a given period of time has lapsed since the tourniquet has first been applied. This alert or signal may be adapted to inform the subject that the application of the tourniquet past a certain period could lead to irreparable damage to the limb or the body of the subject by the tourniquet (where the subject may decide to remove the tourniquet, depending on the seriousness of the wound). The restriction of blood flow caused by the tourniquet could lead to accumulation of metabolites such as potassium, lactate, and myoglobin, etc. which could lead to reperfusion injury. The restriction of blood flow by the tourniquet over time may also result in blood clots (thrombosis), etc. As such, the alert or signal generated by the system 100 informs the subject that past a certain period of application of the tourniquet, the tourniquet may cause irreversible damage to the subject, where the subject may, at that point, assess if the tourniquet is appropriate.

Exemplary Network Architecture for Monitoring Pressure Applied by a Tourniquet to a Limb of a Patient

Reference is made to FIG. 2B, illustrating an exemplary network architecture for monitoring pressure applied by a tourniquet to a limb of a patient. In some embodiments, the monitoring system 100 may communicate historical pressure-time data to the healthcare provider over the network. However, in other implementations, the monitoring system 100 may communicate the historical pressure-time data to the healthcare provider via a short-range wireless connection (e.g. a Bluetooth connection) when the monitoring system 100 is in proximity with the external computing device 158 of the healthcare provider. In other embodiments, the healthcare provider, receiving the patient with the monitoring system 100, may provide user input on the user input interface 157 of the monitoring system 100, e.g. for causing a display of information on the pressure, based from the historical pressure-time data, applied to the limb of the patient appearing on the display 153 of the monitoring system 100.

The external computing device 158 may be a laptop computer, a desktop computer, a smartphone, a smartwatch, a tablet computer, etc.

The external computing device 158 may run a web-based application program, causing a display of a graphical user interface on a display of the external computing device 158, to show information on the pressure applied to the limb by the tourniquet over a period of time.

The network architecture may include one or more monitoring systems 100 that are communicating wirelessly with one or more servers 160 (over an Internet connection, a local area network, etc.), and one or more external computing devices 158.

The monitoring system 100 may transmit pressure readings and time readings for storage in server 160 (e.g. along with metadata providing information on the patient receiving the tourniquet 103 (such as a name, age, gender, weight, height, etc.), an identifier on the monitoring system 100 and/or the tourniquet 103, etc.) The pressure readings and time readings may be stored on the server 160 (e.g. as part of a data structure for that a patient, corresponding to a patient identifier, a monitoring system 100, corresponding to the identifier of the monitoring system 100, etc.) a request may be sent by an external computing device 158 to the server 160 to retrieve historical pressure-time data corresponding to a patient and/or a monitoring device 100, for evaluating if the tourniquet was properly applied to the patient. The external computing device 158 may perform a call for an application programming interface (API) in order to access the server 160.

In some instances, the monitoring system 100 may transmit the pressure readings and time readings directly to the external computing device 158 (or as a dataset of historical time-pressure data following a request generated at the external computing device or user input received at the monitoring system 100 regarding the transmission of the historical time-pressure data).

### Exemplary Method of Monitoring Pressure Applied by a Tourniquet to a Limb of a Patient and of Transmitting Information on the Applied Pressure to a Healthcare Provider

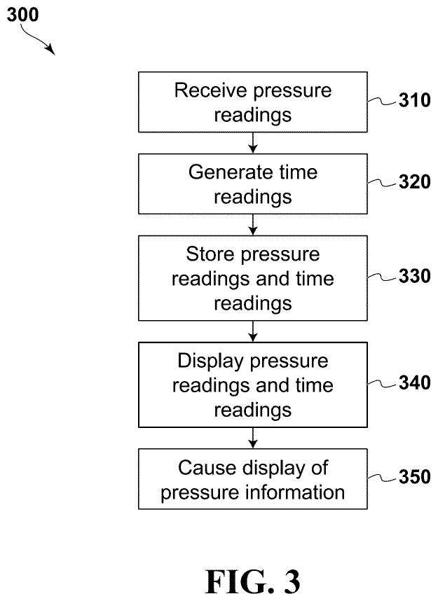

Reference is now made to FIG. 3, illustrating an exemplary method 300 of monitoring pressure applied by a tourniquet to a limb of a patient and of transmitting information on the applied pressure to that limb to a healthcare provider. Monitoring system 100 may be used to perform method 300. However, it will be understood that any other pressure monitoring system in accordance with the present teachings may be used.

Following application of a tourniquet to a limb, a pressure sensor may be inserted between the limb and the tourniquet. As such, the tourniquet also applies pressure to the pressure sensor (e.g. caught between the tourniquet and the limb, or joined to a computing device of the monitoring system), and can measure the pressure or force applied by the tourniquet to the limb.

The pressure readings generated by the pressure sensor are received at step 310. The pressure readings may be transmitted via a wired connection connecting the pressure sensor to the computing device that receives the readings. In some instances, the pressure readings may be transmitted wirelessly. The pressure readings may be taken and transmitted punctually (after a lapse of a given period of time), or may be taken continuously.

Time readings are generated (and/or received) at step 320 (e.g. generated by a clock or timer), following a trigger event. The trigger event may cause the beginning of taking the time readings. For instance, the trigger event may be a detection that tension is being applied to the tourniquet (e.g. based from a pressure reading generated by the pressor sensor, or a sensor connected to the tourniquet), receiving user input at the timer user input interface 162, a turning on of the computing device of the pressure monitoring system that receives the pressure readings from the pressure sensor, etc.

In some instances, input may be received to stop generating pressure readings by the pressure sensor. For example, user input (e.g. a button press) may be received at a user input interface for stopping generating the pressure readings. For example, a reading that no pressure is being applied to the pressure sensor may act as an indicator that the tourniquet has released the limb, and a reading of no pressure at the pressure sensor may result in a command to stop generating pressure readings at the pressure sensor.

The pressure readings and time readings are stored in memory at step 330. The pressure readings and time readings may be stored locally, in the memory of the pressure monitoring system with the pressure sensor that is generating the pressure readings. In some embodiments, the pressure readings and time readings may be stored externally, e.g. following transmission to an external server. The pressure readings and the time readings may be stored as or as part of a data structure associated to a patient profile or to the monitoring system generating the pressure readings and time readings. The time readings and pressure readings may be transmitted with metadata (e.g. information on an identifier of the patient, information on an identifier of the monitoring system, characteristic values for the patient, related to age, gender, weight, height, etc. of the patient). In some instances, geographical information on a location of the pressure monitoring system (e.g. coordinates generated by a global positioning system (GPS) of the pressure monitoring system) may also be stored in memory. The historical or past pressure readings, and the corresponding time readings, are referred to herein as historical pressure-time data.

In some instances, the pressure readings and time readings, or information corresponding to the pressure readings and time readings, may be displayed on a display of the pressure monitoring system at step 340. For instance, the pressure readings and time readings may be displayed in real time as values, in order to give the first responder securing the tourniquet to the limb of the patient an indication regarding the pressure applied by the tourniquet to the limb, and/or to the elapsed time during which the tourniquet is secured around the limb of the patient. In some instances, information derived from the pressure readings and time readings may be displayed, such as a graph showing a change in pressure applied by the tourniquet to the limb over time, or an average value of the pressure applied to the limb by the tourniquet during a period of time.

In some instances, such as when the pressure monitoring system does not include a display, the pressure monitoring system may generate a signal when a target or threshold pressure value of the pressure applied by the tourniquet to the limb is reached. For instance, the signal may be a sound generated by a speaker of the pressure monitoring system, a vibration generated by one or more motors of the pressure monitoring system, etc. When the pressure monitoring system includes a display, a message or icon may be displayed on the display of the pressure monitoring system indicative that the target pressure or pressure threshold has been reached. Information may also be stored in memory of the pressure monitoring system that the target pressure (associated with a timestamp, in accordance with the relevant time reading) regarding when the target pressure or pressure threshold value has been reached. A time period may also be calculated from when the target pressure or pressure threshold value has been reached, e.g. a value corresponding from this time period stored in memory.

In some instances, the pressure readings and time readings may be displayed (e.g. in real time) on a display of an external device (e.g. a smartphone, a laptop computer, a tablet, etc.) that is in communication with the pressure monitoring system.

Upon the patient being transferred to the healthcare provider (e.g. from the first responder) who will make a decision regarding the state of the limb of the patient and what medical procedure to perform on the limb, information on the pressure applied historically by the tourniquet to the limb (e.g. since the securing of the tourniquet to the limb) is received and displayed (e.g. at a computer of the healthcare provider, or on a display of the pressure monitoring system) at step 350. The displayed pressure information corresponds to (is generated from) the historical pressure-temperature data generated by the pressure monitoring system for the related tourniquet.

The information on the pressure displayed for the healthcare provider acts as an indication regarding if the tourniquet was properly applied to the limb (e.g. that appropriate pressure, based on applicable medical standards, was applied to the limb by the tourniquet).

The information on the pressure may be displayed as one or more of a graph of pressure values over time, as a value of average pressure applied over a period of time, a time value indicative of a duration of time that pressure has been applied by the tourniquet to the limb, etc.

The display of information may occur after a trigger event. For instance, the trigger event may be an operator providing user input at a user input interface (e.g. pressing a button) of the pressure monitoring system for causing a display of information related to the historical pressure-time data on the display of the pressure monitoring system or on the display of the external computing device that is in communication with the pressure monitoring system. The trigger event may be the pressure monitoring system being in proximity of an external computing device on which the information is to be displayed (e.g. the pressure monitoring system is in sufficient range with the external computing device to establish a short-range wireless connection, such as a Bluetooth connection, between the pressure monitoring system and the external computing device). A security authorization, such as a handshake, may occur between the pressure monitoring system and the external computing device, prior to the transmission of the historical pressure-time data from the pressure monitoring system to the external computing device. In some instances, the trigger event may be receiving at the pressure monitoring system a command or signal generated by the external computing device (via a graphical user interface of an application program running on the external computing device, such as a mouse click of an icon appearing on the graphical user interface) to cause a transmission of the historical pressure-time data to the external computing device for display of the related pressure information.

In some embodiments, the historical time-pressure data is transmitted from the pressure monitoring system to the external computing device via a wired connection. In some instances, the historical time-pressure data may be transmitted to the external computing device via a wireless connection (such as a short-range wireless connection like a Bluetooth connection). In some instances, an RFID tag system may be used for verification of an identity of the pressure monitoring system prior to transmission of the historical pressure-time data to the external computing device.

In some embodiments, user input may be received at the computing device to annotate one or more portions of the data that is displayed (e.g. as a graph) on the display of the computing device, at a given recorded time or time period (e.g. adding a pin or note indicative of a change in a state of the limb of the patient).

In some embodiments, the historical pressure-time data that is the basis for displaying the pressure information is received from an external server, when the pressure monitoring system transmits the generated pressure readings and time readings to the external server for storage of the generated pressure readings and time readings in the external server. The external computing device may query the external server for the historical pressure-time data corresponding to a pressure monitoring system (e.g. a pressure monitoring system that is measuring pressure applied by a tourniquet to a patient of the healthcare provider), using e.g. a unique identifier for the target pressure monitoring system (e.g. transmitted to the external computing device by the pressure monitoring system), an identifier for the patient, etc.

Each of, or a combination of, pressure and time readings may be used to determine limb viability after the application of a tourniquet as treatment for severe hemorrhage. The pressure applied to the limb by the tourniquet may dictate if the limb may eventually need to be amputated, as insufficient pressure applied can decrease the probability of limb viability once surgical operations are able to be performed. In some instances, the duration of time the tourniquet was applied may dictate if the limb may eventually need to be amputated, as prolonged application of a tourniquet may cause damage to the muscle tissue after a few hours without blood flow, irreversible ischemic damage, tissue necrosis, nerve damage, etc. Information on pressure applied to the limb by the tourniquet over time can provide information concerning the viability of the limb and the necessity of amputation of the limb to a healthcare provider.

A pressure reading should be understood to include any measurement taken by a pressure sensor as described herein. This should include pressure values derived from force measurements. Pressure readings can be taken manually or automatically at certain intervals of time.

A time reading should be understood to include any value of time that could be used to give temporal information on an event or another measurement (e.g. pressure reading, etc.). Time readings could indicate an elapsed time since the start of a timer, a date, a time in a local time zone, a period (in hours, minutes, seconds, etc.), etc. Time readings can indicate a passing of time between measurements or events, where a passing of time may be understood to be a period of time elapsed between measurements or between a measurement and an event.

A trigger event should be understood herein as an event which determines the start of generating time readings. The trigger event may be a manual event where a user interacts with a timer user input interface to initiate the start of generating time readings, however the trigger event may also be in relation to an action or change of state detected by a pressure monitoring system such as the application of pressure to a limb by a tourniquet, a determination that the pressure applied to a limb by a tourniquet falling under a set threshold value, a determination that the pressure applied to a limb rise above a set threshold value, etc.

Although the invention has been described with reference to preferred embodiments, it is to be understood that modifications may be resorted to as will be apparent to those skilled in the art. Such modifications and variations are to be considered within the purview and scope of the present invention.

Representative, non-limiting examples of the present invention were described above in detail with reference to the attached drawing. This detailed description is merely intended to teach a person of skill in the art further details for practicing preferred aspects of the present teachings and is not intended to limit the scope of the invention. Furthermore, each of the additional features and teachings disclosed above and below may be utilized separately or in conjunction with other features and teachings.

Moreover, combinations of features and steps disclosed in the above detailed description, as well as in the experimental examples, may not be necessary to practice the invention in the broadest sense, and are instead taught merely to particularly describe representative examples of the invention. Furthermore, various features of the above-described representative examples, as well as the various independent and dependent claims below, may be combined in ways that are not specifically and explicitly enumerated in order to provide additional useful embodiments of the present teachings.

What is claimed is:

1. A pressure monitoring system for recording pressure applied to a limb by a tourniquet, comprising:
a pressure sensor for insertion between the tourniquet and the limb, and for measuring pressure applied by tourniquet to the limb;
a power source;
a processor;
an input/output interface;
memory including program code that, when executed by the processor, causes the processor to:
receive pressure readings from the pressure sensor;
upon receiving a trigger event, generate time readings indicative of a passing of time following the trigger event; and
cause a transmission, via the input/output interface, of the received pressure readings and the time readings for one or more of:
storage of the received pressure readings and the time readings; and
display, on a display, information on pressure applied to the limb by the tourniquet over time related to historical pressure-time data corresponding to a relation between the time readings and the pressure readings over a period of time; and
a housing for containing at least the processor and the memory.

2. The system according to claim 1, wherein the memory further includes program code for causing the processor to:
store the received pressure readings in relation to the generated time readings in the memory as the historical pressure-time data; and
upon an occurrence of a trigger event, cause a transmission, via the input/output interface, of the historical pressure-time data for display on the display of information on pressure applied to the limb by the tourniquet over time related to historical pressure-time data, wherein the causing of a transmission occurs upon an occurrence of a trigger event.

3. The system according to claim 2, further comprising the display.

4. The system according to claim 3, wherein the display is a liquid-crystal display.

5. The system according to claim 3, further comprising a user input interface, wherein the trigger event is receiving input received at the user input interface.

6. The system according to claim 5, wherein the user input interface includes two buttons, wherein a first button of the two buttons is located on a first side of the housing and a second of the two buttons is located on a second side of the housing opposite the first side of the housing, and wherein the trigger event is user input received at the first button and the second button.

7. The system according to claim 1, wherein the input/output interface is configured to establish a wireless connection with the external computing device.

8. The system according to claim 1, wherein the program code further causes the processor to cause a generation of a signal when a target pressure applied by the tourniquet on the limb is reached, determined from the pressure readings received from the pressure sensor.

9. The system according to claim 1, further comprising the tourniquet.

10. The system according to claim 1, further comprising a fastener that is a clip located on the housing for receiving the tourniquet.

11. The system according to claim 10, wherein the clip comprises one or more teeth projecting from a surface of the clip that is facing the housing and that is adapted to contact the tourniquet once the tourniquet is inserted in a space defined between the clip and the housing.

12. The system according to claim 11, wherein the one or more teeth comprises rows of teeth.

13. A method of sharing information on a state of a limb that has undergone ischemia resulting from the application of a tourniquet to the limb, comprising:
upon occurrence of a trigger event, receiving historical pressure-time data generated by a pressure sensor adapted to generate pressure readings of pressure applied from the tourniquet to the limb, the historical pressure-time data indicative of pressure applied over a period of time by the tourniquet, wherein the historical pressure-time data provides information on the state of the limb based on a sufficiency of pressure applied to the limb by the tourniquet.

14. The method according to claim 13, wherein the trigger event is a reception or transmission of user input for causing transmission of the received historical pressure-time data.

15. The method according to claim 13, wherein the information on the state of the limb is displayed as a graph corresponding to the historical pressure-time data.

16. The method according to claim 13, further comprising fastening the pressure sensor to the tourniquet using a clip.

17. The method according to claim 16, wherein the clip comprises teeth for digging into the tourniquet.

18. A method of sharing information on a state of a limb that has undergone ischemia resulting from the application of a tourniquet to the limb, comprising:
upon occurrence of a trigger event, causing a display of information indicative of the state of the limb based on a sufficiency of pressure applied to the limb by the tourniquet over a period of time, the information derived from historical pressure-time data generated by a pressure sensor adapted to generate pressure readings of pressure applied from the tourniquet to the limb to the historical pressure-time data indicative of pressure applied over the period of time by the tourniquet.

19. The method according to claim 18, wherein the information is displayed on a display connected to the tourniquet.

20. The method according to claim 18, further comprising transmitting the historical pressure-time data to an external computer following the trigger event, and wherein the information is displayed on a display of the external computer.
